(19) United States
(12) Patent Application Publication
(10) Pub. No.: US 2021/0318265 A1
(43) Pub. Date: Oct. 14, 2021

(54) POLYMERASE ATTACHMENT TO A CONDUCTIVE CHANNEL

(71) Applicant: ILLUMINA, INC., San Diego, CA (US)

(72) Inventors: Yannan ZHAO, San Diego, CA (US); Emily WELCH, San Diego, CA (US)

(73) Assignee: ILLUMINA, INC., San Diego, CA (US)

(21) Appl. No.: 17/256,695

(22) PCT Filed: Jun. 12, 2020
(86) PCT No.: PCT/US2020/037389

§ 371 Date: Dec. 29, 2020

Related U.S. Application Data
(60) Provisional application No. 62/862,767, filed on Jun. 18, 2019.

(51) Int. Cl. G01N 27/414; C12Q 1/6869

(52) U.S. Cl. CPC G01N 27/4145; G01N 27/4146; C12Q 1/6869

(57) ABSTRACT

Provided is a device, including a conductive channel and a number of polymerase molecules bound thereto, wherein the number is between one and five and the conductive channel is to detect incorporation of a nucleotide comprising a charge tag into a nascent polynucleotide by the polymerase, and each of the one or more polymerase molecules includes a hexahistidine tag, the conductive channel includes a multivalent nickel-nitrolotriacetic acid complex, and the hexahistidine tag is bound to the multivalent nickel-nitrolotriacetic acid complex. Also provided are a method of attaching a polymerase to a conductive channel of a device and a method of using the device.

### CROSS-REFERENCE TO RELATED APPLICATIONS

This application claims priority to U.S. Provisional Patent Application No. 62/862,767, filed Jun. 18, 2019, the entire contents of which are hereby incorporated herein.

### BACKGROUND

The majority of current sequencing, genotyping, and related platforms use “sequencing by synthesis” (SBS) technology and fluorescence based methods for detection. Alternative sequencing methods that allow for more cost effective, rapid, and convenient sequencing and nucleic acid detection are desirable as complements to SBS. Charge based sequencing is an attractive approach. For related methods and systems, an ability to controllably bind a polymerase to, and release a polymerase from binding to, a substrate, such as a substrate to detect nucleotide incorporation, may be beneficial.

### SUMMARY

In an aspect, provided is a device, including a conductive channel and a number of polymerase molecules attached to said conductive channel, wherein the number is between one and five and the conductive channel is to detect incorporation of a nucleotide including a charge tag into a nascent polynucleotide by the polymerase, and each of the one or more polymerase molecules includes a histidine tag, the conductive channel includes a nickel-nitrolotriacetic acid complex, and the histidine tag is bound to the nickel-nitrolotriacetic acid complex.

In an example, the number of polymerase molecules bound to said conductive channel is five or fewer, such as five, four, three two, or one. In a different example, the number may be more than five. In another example, the nickel-nitrolotriacetic acid complex includes nine nickel-nitrolotriacetic acid groups. In yet another example, the conductive channel includes a nanowire having a diameter of between about 10 nm and about 100 nm and a length of between about 50 nm and about 300 nm. In still another example, the nanowire has a diameter of about 30 nm and a length of between about 100 nm and about 150 nm. In a further example, a surface of the conductive channel further includes a plurality of polyethylene glycol moieties not directly bound to a complex of nitrolotriacetic acid groups.

In another aspect, provided is a method, including attaching between one and five nickel-nitrolotriacetic acid complexes to a conductive channel and attaching a polymerase including a histidine tag to one or more of the nickel-nitrolotriacetic acid complexes, wherein the conductive channel is to detect incorporation of a nucleotide comprising a charge tag into a nascent polynucleotide by the polymerase.

In an example, the number of polymerase molecules attached to said conductive channel is five or fewer, such as five, four, three two, or one. In a different example, the number may be more than five. In another example, the nickel-nitrolotriacetic acid complex includes nine nickel-nitrolotriacetic acid groups. In yet another example, the conductive channel includes a nanowire having a diameter of between about 10 nm and about 100 nm and a length of between about 50 nm and about 300 nm. In still another example, the nanowire has a diameter of about 30 nm and a length of between about 100 nm and about 150 nm. In a further example, a surface of the conductive channel further includes a plurality of polyethylene glycol moieties not directly bound to a complex of nitrolotriacetic acid groups. In yet a further example, the method further includes eluting the polymerase from the between one and five nickel-nitrolotriacetic acid complexes, wherein eluting includes chelating nickel with ethylenediaminetetraacetic acid or imidazole. In still a further example, the method further includes reloading the nitrolotriacetic acid moieties with nickel to re-form nickel-nitrolotriacetic acid complexes and binding a polymerase to the re-formed nickel-nitrolotriacetic acid complexes.

In yet another aspect, provided is a method, including detecting incorporation, using a number of polymerases, of one or more nucleotides into one or more nascent polynucleotide strands complementary to one or more template polynucleotide strands, wherein the one or more polymerases are each attached to a conductive channel, one or more of the one or more nucleotides comprises a charge tag and the conductive channel is to detect the charge tag during the incorporation, wherein each of the one or more polymerases includes a histidine tag, the conductive channel includes a nickel-nitrolotriacetic acid complex, and the histidine tag is bound to the nickel-nitrolotriacetic acid complex.

In an example, the number of polymerase molecules attached to said conductive channel is five or fewer, such as five, four, three two, or one. In a different example, the number may be more than five. In another example, the nickel-nitrolotriacetic acid complex includes nine nickel-nitrolotriacetic acid groups. In yet another example, the conductive channel includes a nanowire having a diameter of between about 10 nm and about 100 nm and a length of between about 50 nm and about 300 nm. In still another example, the nanowire has a diameter of about 30 nm and a length of between about 100 nm and about 150 nm. In a further example, surface of the conductive channel further includes a plurality of polyethylene glycol moieties not directly bound to a complex of nitrolotriacetic acid groups.

### BRIEF DESCRIPTION OF THE DRAWINGS

These and other features, aspects, and advantages of the present disclosure will become better understood when the following detailed description is read with reference to the accompanying drawings, wherein:

FIG. 1 shows a polymerase attached to a charge sensor.

FIG. 2 shows Ni-NTA attached to a polyhistidine tag.

FIG. 3 shows an example of an attachment of an NTA group to a surface.

FIG. 4 shows an example of a process for attaching an NTA group to a surface.

FIG. 5A and FIG. 5B show examples of workflows for methods in accordance with aspects of the present disclosure.

FIG. 6 is a graph representing measurements taken of histidine-tagged protein attached to a surface with Ni-NTA complexes as attachment moieties.

### DETAILED DESCRIPTION

There is presently a need for improved devices, systems, and methods for real-time detection of nucleotides incorporated by polymerases in such methods. An attractive option is detecting polymerase-mediated nucleotide incorporation by using a conductive channel, wherein a polymerase is tethered to a conductive channel and the nucleotide includes a charge-bearing tag. During pairing of the nucleotide with a complementary template nucleotide during polymerase-mediated incorporation, the conductive channel detects the presence of the charge-bearing tag of the incorporated nucleotide and, thereby, the identity of the incorporated nucleotide.

Disclosed herein is a method for controlling the number of polymerase molecules tethered to a given conductive channel. The disclosure provides controllably tethering a low enough number of polymerases to a conductive channel to avoid detection of too many, including multiple, disparate, nucleotide incorporation events. Also provided is the tethering of a polymerase to a conductive channel with sufficient bond strength such that a polymerase may stay bound to a conductive channel for however so long is desired.

This disclosure provides examples of immobilization or attachment of a nucleotide polymerase to a surface of a device for detection of incorporation of a nucleotide into a nascent nucleotide strand or a primer complementary to a template for determining the identity of one or more nucleotides in the template. In one example, attachment is sufficiently strong or enduring so as to prevent unwanted detachment of a polymerase from the substrate, such as during one or more processes as part of a sequencing, genotyping, or related method for identifying one or more nucleotides in a template. Attachment may also be reversible. For example, if a fidelity, selectivity, efficiency, polymerase activity, or other features of a polymerase deteriorates or is at risk of deteriorating, a polymerase may, as disclosed herein, be controllably released from the substrate and a new polymerase attached to the substrate.

As also disclosed herein, the number of polynucleotides immobilized or attached to a substrate may be controlled. In some sequencing by synthesis or related methods, a pre-determined, low, or otherwise generally controlled number of polymerase molecules attached or connected to a substrate is obtained as disclosed herein. For example, in some sequencing by synthesis or related methods, the identity of a nucleotide in a template molecule (for example, whether it includes adenine, guanine, cytosine, thymine, uracil, as its nitrogenous base) is determined by identifying a nucleotide complementary thereto incorporated into a nascent polynucleotide strand or primer, hybridized to the template, by a polymerase. In turn, nucleotides for addition to a nascent strand or template by a polymerase may carry or include a mark or tag signifying their identity. Detection of a nucleotide incorporated by a polymerase thereby indirectly indicates the identity of the complementary nucleotide of the template.

For example, nucleotides incorporated by a polymerase may contain or include a tag that carries a charge, or a charge tag. When such a charge-tagged nucleotide is brought together with its complementary base in a template by a polymerase during polymerization, the charge tag may be sensed by the substrate to which the polymerase is immobilized or attached, wherein the substrate includes a conductive channel. A conductive channel for detecting a modified nucleotide including a charge may be responsive to a surrounding electric field. This field is modulated by positioning a modified nucleotide with a charge close proximity to a surface of the conductive channel. Close proximity of the charge tags to the surface may be important in some cases, such as if salt or other ions in the solution may screen a charge from detection by a conductive channel. A characteristic screening length is referred to as a Debye length, beyond which a conductive channel may be unable to detect charge.

For example a conductive channel may be a nanostructured transistor, such as a nanowire or other nano-scaled, charge-gated, electrically semi-conductive nanostructure. A nanowire may have a diameter of between about 10 nm and about 100 nm and a length of between about 50 nm and about 300 nm. For example, a nanowire may have a diameter of about 30 nm and a length of between about 100 nm and about 150 nm, such as about 130 nm. In other examples, a nanowire may be anywhere from 50 nm to 5 μm in length, such as from about 50 nm to about 100 nm, about 50 nm, about 60 nm, about 70 nm, about 80 nm, about 90 nm in length, or about 100 nm in length, or about 100 nm to about 150 nm, about 100 nm, about 110 nm, about 120 nm, about 130 nm, about 140 nm in length, or about 150 nm in length, or about 150 nm to about 200 nm, about 150 nm, about 160 nm, about 170 nm, about 180 nm, about 190 nm in length, or about 200 nm in length, or about 200 nm to about 250 nm, about 200 nm, about 210 nm, about 220 nm, about 230 nm, about 240 nm in length, or about 250 nm in length, or about 250 nm to about 300 nm, about 250 nm, about 260 nm, about 270 nm, about 280 nm, about 290 nm in length, or about 300 nm in length, or about 300 nm to about 350 nm, about 300 nm, about 310 nm, about 320 nm, about 330 nm, about 340 nm in length, or about 350 nm in length, or from about 350 nm to about 400 nm, about 350 nm, about 360 nm, about 370 nm, about 380 nm, about 390 nm in length, or about 400 nm in length, or from about 400 nm to about 500 nm, about 400 nm, about 410 nm, about 420 nm, about 430 nm, about 440 nm in length, or about 450 nm in length, or from about 500 nm to about 750 nm, about 500 nm, about 550 nm, about 600 nm, about 650 nm, about 700 nm in length, or about 750 nm in length, or from about 750 nm to about 1 μm, about 750 nm, about 800 nm, about 850 nm, about 900 nm, about 950 nm in length, or about 1 μm in length, or from about 1 μm to about 1.5 μm, about 1 μm, about 1.1 μm, about 1.2 μm, about 1.3 μm, about 1.4 nm in length, or about 1.5 μm in length, or from about 1.5 μm to about 2 μm, about 1.5 μm, about 1.6 μm, about 1.7 μm, about 1.8 μm, about 1.9 nm in length, or about 2.0 μm in length, or from about 2.0 μm to about 2.5 μm, about 2.0 μm, about 2.1 μm, about 2.2 μm, about 2.3 μm, about 2.4 nm in length, or about 2.5 μm in length, or from about 2.5 μm to about 3 μm, about 2.5 μm, about 2.6 μm, about 2.7 μm, about 2.8 μm, about 2.9 nm or about 3.0 μm in length, or from about 3.0 μm to about 3.5 μm, about 3.0 μm, about 3.1 μm, about 3.2 μm, about 3.3 μm, about 3.4 nm or about 3.5 μm in length, or from about 3.5 μm to about 4.0 μm, about 3.5 μm, about 3.6 μm, about 3.7 μm, about 3.8 μm, about 3.9 nm or about 4.0 μm in length, or from about 4 μm to about 5 μm, about 4.0 μm, about 4.2 μm, about 4.4 μm, about 4.6 μm, about 4.8 nm or about 5.0 μm in length, or longer or shorter, or any length within or between these ranges.

Transient presence of a charge tag during polymerization by the polymerase may be detected by the conductive channel, controlling the flow of current therethrough. In some examples, different nucleotides including different nitrogenous bases from each other may include charge tags whose charge differs from each other such that a conductive channel may respond differently to the presence of nucleotides containing nitrogenous bases that differ from each other.

As used herein, the term “attached” or “bound” refers to the state of two things being joined, fastened, adhered, or connected to each other. For example, a reaction component, such as a polymerase, can be attached or bound to a solid phase component, such as a conductive channel, by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions and hydrophobic interactions. Two things may be attached or bound to each other reversibly, meaning the attachment or bond between them may be formed then subsequently broken or disrupted, then optionally reformed, perhaps after replacement of one of the two things with another thing. In the case of non-covalent attachment, in some examples the binding of two things to each other may require presence of another factor such as one or more metal or other ion to permit binding of the two things together. In such examples, the attachment or bond may be reversible in that removal or chelation of said one or more metal or other ion may result in detaching the two things from each other. Subsequently, when presence of the one or more metal or other ion is restored, the two things may again become attached or bounds to each other.

As used herein, the term “electrically conductive channel” is intended to mean a portion of a detection device that translates perturbations at its surface or in its surrounding electrical field into an electrical signal. The conductive channel may be an electrically conductive channel.

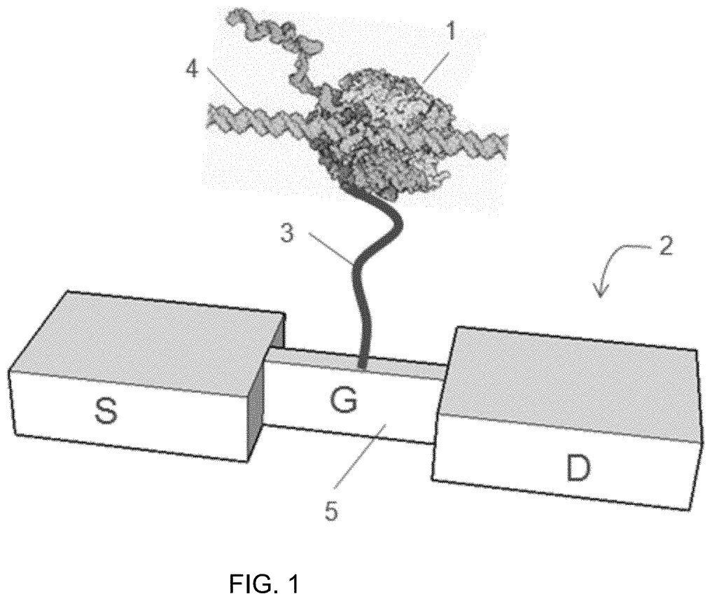

A non-limiting example of a polymerase attached to a charge sensor is shown in FIG. 1. Briefly, polymerase 1 may be immobilized on the gate 5 of a silicon nanowire field-effect transistor (FET) 2 with a tether 3. Optionally, the nanowire can be made of material other than silicon or the nanowire can be replaced with a nanotube. An example of a template is ssDNA 4 to be sequenced is bound to polymerase 1 after having been introduced in solution along with nucleotides and other reactants. As the complimentary strand is synthesized, disturbances in the charge distribution in the vicinity of the FET 2 are generated, either as a result of conformational changes of the polymerase 1, or due to presence of the nucleotides, possibly modified with an electrically active tag in the vicinity of the FET 2. Those modifications in the charge distribution are sensed by the nanowire-FET 2 and detected as a modulation in the FET transconductance current. The electrically conductive channel 5 may be the channel of a conductive channel 2. The conductive channel 2 may include source and drain terminals S, D and the channel 5 connecting the terminals S, D. The channel may have any suitable geometries—e.g., tube, wire, plate, etc.

As used herein, the term “conductive channel” is intended to mean a detection device that translates perturbations at its surface or in its surrounding electrical field into an electrical signal. For example, a conductive channel can translate the arrival or departure of a reaction component into an electrical signal. A conductive channel can also translate interactions between two reaction components, or conformational changes in a single reaction component, into an electrical signal. A conductive channel may have any suitable geometries. For example, the channel may be a nanotube, a nanowire, a nanoribbon, etc. The conductive channel may comprise any suitable electrically conductive material. The conductive material may comprise an organic material, an inorganic material, or both. For example, the channel may comprise a semiconductor. In one example, the channel comprise carbon. In another example, the channel comprise silicon. An example conductive channel is a field effect transistor (FET) such as a carbon nanotube (CNT), single-walled carbon nanotube (SWNT) based FET, silicon nanowire (SiNW) FET, graphene nanoribbon FET (and related nanoribbon FETs fabricated from 2D materials such as MoS₂, silicene, etc.), tunnel FET (TFET), and steep subthreshold slope devices (see, for example, Swaminathan et al., Proceedings of the 51st Annual Design Automation Conference on Design Automation Conference, pg 1-6, ISBN: 978-1-4503-2730-5 (2014) and Ionescu et al., Nature 479, 329-337 (2011); each of which is incorporated by reference in its entirety). Examples of FET and SWNT conductive channels that can be used in the methods and apparatus of the present disclosure are set forth in US Pat. App. Pub. No. 2013/0078622 A1, which is incorporated herein by reference in its entirety.

The terminals S, D of FIG. 1 may comprise any suitable electrically conductive material. Examples of suitable source and drain materials include cobalt, cobalt silicide, nickel, nickel silicide, aluminum, tungsten, copper, titanium, molybdenum, indium tin oxide (ITO), indium zin oxide, gold, platinum, carbon, etc.

The conductive channel 5 may include any conductive or semi-conductive material that can oxidize or reduce the redox-active charge tag. The material may comprise an organic material, an inorganic material, or both. Some examples of suitable channel materials include silicon, carbon (e.g., glassy carbon, graphene, etc.), polymers, such as conductive polymers (e.g., polypyrrole, polyaniline, polythiophene, poly(3,4-ethylenedioxythiophene) doped with poly(4-styrenesulfonate) (PEDOT-PSS), etc.), metals, biomolecules, etc. Electrically conductive channel 5 can translate the arrival or departure of a reaction component (e.g., a labeled nucleotide) into an electrical signal. In examples disclosed herein, electrically conductive channel 5 can also translate interactions between two reaction components (the template nucleic acid and a nucleotide of the labeled nucleotide) into a detectable signal through its interaction with the redox-active charge tag of the labeled nucleotide.

In some examples, conductive channel 5 may also be a nanostructure that has at least one dimension on the nanoscale (ranging from 1 nm to less than 1 μm). In one example, this dimension refers to the largest dimension. As examples, the electrically conductive channel 5 may be a semi-conducting nanostructure, a graphene nanostructure, a metallic nanostructure, and a conducting polymer nanostructure. The nanostructure may be a multi- or single-walled nanotube, a nanowire, a nanoribbon, etc.

In particular examples, an apparatus or method of the present disclosure may use deeply scaled FinFET transistors as single-molecule conductive channels. FinFET conductive channels benefit from technology already under development by leading edge semiconductor manufacturers. Furthermore, previously published components can be used, including but not limited to (1) those used for immobilization of lysozyme on CNT to observe enzyme processivity in real time as described in Choi et al, Science, 335, 319 (2012), (2) those used to immobilize the Pol 1 Klenow fragment on CNT and observe DNA processivity in real time as described in Olsen et al, J. Amer. Chem. Soc., 135, 7885 (2013), (3) those used to elucidate a transduction mechanism as moving charged residues due to protein allosteric motion as described in Chi et al, NanoLett 13, 625 (2013). The present methods can also employ the apparatus, components of the apparatus, and methods set forth in US Pat. App. Pub. No. 2013/0078622 A1. Each of the above references is incorporated herein by reference in its entirety.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “about” when used in reference to a number, dimension, or measurement includes a value that may vary from the recited numeral that follows by up to 5%, such that, for example, “about 100” would mean “from 95 to 105”.

As used herein, the term “label,” when used in reference to a reaction component, is intended to mean a detectable reaction component or detectable moiety of a reaction component. A useful label is a charge label (also called a charge tag) that can be detected by a conductive channel. A label can be intrinsic to a reaction component that is to be detected (e.g. a charged amino acid of a polymerase) or the label can be extrinsic to the reaction component (e.g. a non-naturally occurring modification of an amino acid). In some examples a label can include multiple moieties having separate functions. For example a label can include a linker component (such as a nucleic acid) and a charge tag component.

As used herein, the term “nucleic acid” is intended to be consistent with its use in the art and includes naturally occurring nucleic acids or functional analogs thereof. Particularly useful functional analogs are capable of hybridizing to a nucleic acid in a sequence specific fashion or capable of being used as a template for replication of a particular nucleotide sequence. Naturally occurring nucleic acids generally have a backbone containing phosphodiester bonds. An analog structure can have an alternate backbone linkage including any of a variety of those known in the art such as peptide nucleic acid (PNA) or locked nucleic acid (LNA). Naturally occurring nucleic acids generally have a deoxyribose sugar (e.g. found in deoxyribonucleic acid (DNA)) or a ribose sugar (e.g. found in ribonucleic acid (RNA)).

A nucleic acid can contain any of a variety of analogs of these sugar moieties that are known in the art. A nucleic acid can include native or non-native bases. In this regard, a native deoxyribonucleic acid can have one or more bases selected from the group consisting of adenine, thymine, cytosine, or guanine and a ribonucleic acid can have one or more bases selected from the group consisting of uracil, adenine, cytosine or guanine. Useful non-native bases that can be included in a nucleic acid are known in the art.

As used herein, the term “nucleotide” is intended to include natural nucleotides, analogs thereof, ribonucleotides, deoxyribonucleotides, dideoxyribonucleotides and other molecules known as nucleotides. The term can be used to refer to a monomeric unit that is present in a polymer, for example to identify a subunit present in a DNA or RNA strand. The term can also be used to refer to a molecule that is not necessarily present in a polymer, for example, a molecule that is capable of being incorporated into a polynucleotide in a template dependent manner by a polymerase. The term can refer to a nucleoside unit having, for example, 0, 1, 2, 3 or more phosphates on the 5′ carbon. For example, tetraphosphate nucleotides, pentaphosphate nucleotides, and hexaphosphate nucleotides can be particularly useful, as can nucleotides with more than 6 phosphates, such as 7, 8, 9, 10, or more phosphates, on the 5′ carbon. Example natural nucleotides include, without limitation, ATP, UTP, CTP, and GTP (collectively NTP), and ADP, UDP, CDP, and GDP (collectively NDP), or AMP, UMP, CMP, or GMP (collectively NMP), or dATP, dTTP, dCTP, and dGTP (collectively dNTP), and dADP, dTDP, dCDP, and dGDP (collectively dNDP), and dAMP, dTMP, dCMP, and dGMP (dNMP). Example nucleotides may include, without exception, any NMP, dNMP, NDP, dNDP, NTP, dNTP, and other NXP and dNXP where X represents a number from 2 to 10 (collectively NPP).

Non-natural nucleotides also referred to herein as nucleotide analogs, include those that are not present in a natural biological system or not substantially incorporated into polynucleotides by a polymerase in its natural milieu, for example, in a non-recombinant cell that expresses the polymerase. Particularly useful non-natural nucleotides include those that are incorporated into a polynucleotide strand by a polymerase at a rate that is substantially faster or slower than the rate at which another nucleotide, such as a natural nucleotide that base-pairs with the same Watson-Crick complementary base, is incorporated into the strand by the polymerase. As examples of such substantially faster or lower rates, a non-natural nucleotide may be incorporated at a rate that is at least about 2 fold different—e.g., at least about 5 fold different, about 10 fold different, about 25 fold different, about 50 fold different, about 100 fold different, about 1000 fold different, about 10000 fold different, or more when compared to the incorporation rate of a natural nucleotide. A non-natural nucleotide can be capable of being further extended after being incorporated into a polynucleotide. Examples include, nucleotide analogs having a 3′ hydroxyl or nucleotide analogs having a reversible terminator moiety at the 3′ position that can be removed to allow further extension of a polynucleotide that has incorporated the nucleotide analog. Examples of reversible terminator moieties that can be used are described, for example, in U.S. Pat. Nos. 7,427,673; 7,414,116; and 7,057,026 and PCT publications WO 91/06678 and WO 07/123744, each of which is incorporated herein by reference in its entirety. It will be understood that in some examples a nucleotide analog having a 3′ terminator moiety or lacking a 3′ hydroxyl (such as a dideoxynucleotide analog) can be used under conditions where the polynucleotide that has incorporated the nucleotide analog is not further extended. In some examples, nucleotide(s) may not include a reversible terminator moiety, or the nucleotides(s) will not include a non-reversible terminator moiety or the nucleotide(s) will not include any terminator moiety at all. Nucleotide analogs with modifications at the 5′ position are also useful.

As used herein, the term “reaction component” is intended to mean a molecule that takes part in a reaction. Examples include, reactants that are consumed in a reaction, products that are created by a reaction, catalysts such as enzymes that facilitate a reaction, solvents, salts, buffers and other molecules.

As used herein, the term “terminator moiety,” when used in reference to a nucleotide, means a part of the nucleotide that inhibits or prevents the nucleotide from forming a covalent linkage to a second nucleotide. For example, in the case of nucleotides having a pentose moiety, a terminator moiety can prevent formation of a phosphodiester bond between the 3′ oxygen of the nucleotide and the 5′ phosphate of the second nucleotide. The terminator moiety can be part of a nucleotide that is a monomer unit present in a nucleic acid polymer or the terminator moiety can be a part of a free nucleotide (e.g. a nucleotide triphosphate). The terminator moiety that is part of a nucleotide can be reversible, such that the terminator moiety can be modified to render the nucleotide capable of forming a covalent linkage to a second nucleotide. In particular examples, a terminator moiety, such as a reversible terminator moiety, can be attached to the 3′ position or 2′ position of a pentose moiety of a nucleotide analog.

Any of a variety of polymerases can be used in a method or composition set forth herein including, for example, protein-based enzymes isolated from biological systems and functional variants thereof. Reference to a particular polymerase, such as those exemplified below, will be understood to include functional variants thereof unless indicated otherwise. A particularly useful function of a polymerase is to catalyze the polymerization of a nucleic acid strand using an existing nucleic acid as a template. Other functions that are useful are described elsewhere herein. Examples of useful polymerases include DNA polymerases and RNA polymerases, functional fragments thereof, and recombinant fusion peptides including them. Example DNA polymerases include those that have been classified by structural homology into families identified as A, B, C, D, X, Y, and RT. DNA Polymerases in Family A include, for example, T7 DNA polymerase, eukaryotic mitochondrial DNA Polymerase gamma., E. coli DNA Pol I (including Klenow fragment), Thermus aquaticus Pol I, and Bacillus stearothermophilus Pol I. DNA Polymerases in Family B include, for example, eukaryotic DNA polymerases a, 6, and E; DNA polymerase C; T4 DNA polymerase, Phi29 DNA polymerase, 9° NTM, and RB69 bacteriophage DNA polymerase. Family C includes, for example, the E. coli DNA Polymerase III alpha subunit. Family D includes, for example, polymerases derived from the Euryarchaeota subdomain of Archaea. DNA Polymerases in Family X include, for example, eukaryotic polymerases Pol beta, Pol sigma, Pol lamda, and Pol mu, and S. cerevisiae Pol4. DNA Polymerases in Family Y include, for example, Pol eta, Pol iota, Pol kappa, E. coli Pol IV (DINB) and E. coli Pol V (UmuD′2C). The RT (reverse transcriptase) family of DNA polymerases includes, for example, retrovirus reverse transcriptases and eukaryotic telomerases. Example RNA polymerases include, but are not limited to, viral RNA polymerases such as T7 RNA polymerase; Eukaryotic RNA polymerases such as RNA polymerase I, RNA polymerase II, RNA polymerase III, RNA polymerase IV, and RNA polymerase V; and Archaea RNA polymerase. Other polymerases, disclosed in U.S. Pat. No. 8,460,910, which is incorporated herein in its entirety, are also included among polymerases as referred to herein, as are any other functional polymerases including those having sequences modified by comparison to any of the above mentioned polymerase enzymes, which are provided merely as a listing of non-limiting examples.

It may be desirable to form an attachment between a polymerase and a conductive channel that is strong enough to sustain repeated washing or processing steps during a sequencing or other reaction, such as during the administration and removal of solutions containing various reaction reagents in succession. Various applications of SBS and related technologies employ movement of a solid state substrate and solution bearing reagents, and/or microfluidic, positive or negative pressure flow, passive flow, or other fluid movements of reagents in solution in order to support binding of reagents to appropriate sites, thorough distribution or removal of buffer, reaction components, reagents, and other compounds during reagent application and washing steps. Attachment of a polymerase to a conductive channel may advantageously be sufficiently stable to withstand such motion of a conductive channel relative to a reaction solution, or mere passage of time, without the polymerase becoming detached from a conductive channel.

As disclosed herein, a polymerase may be modified by the addition of a binding moiety and an attachment moiety may be bound to the conductive channel, such that the polymerase may become attached or bound to the conductive channel. An attachment between a binding moiety and an attachment moiety may be suitably strong to withstand potential disruptions as disclosed above. In some examples, a binding moiety may include a polymer or other repeat of one or more subunits wherein the polymer binds more strongly or effectively to the attachment moiety than does the monomer. In further examples, a polymerase may include multiple polymers forming a binding moiety complex. Similarly, an attachment moiety may include a chemical composition capable of forming an attachment with the binding moiety of the polymerase. The chemical composition may include a collection of functional groups that together can attach or bond to the binding moiety. In some examples, the conductive channel may include a complex of multiple attachment moieties, such as multiple copies of a single attachment moiety, to strengthen or enhance binding to a polymerase's binding moiety.

In some examples, attachment or bonding between one or more attachment moieties of a conductive channel and one or more binding moieties of a polymerase may be enhanced with or require the presence of a metal or other ion or binding cofactor, without which the polymerase and conductive channel would attach or bind only weakly to each other or not at all. In such examples, such a binding cofactor may be added to a conductive channel and polymerase such that their attachment moieties and binding moieties may attach to one another. Subsequently, if removal of the polymerase from the conductive channel is desired, the metal or other ion or binding cofactor may be removed, thereby breaking or severing the attachment between the conductive channel and the polymerase. For example, a chelating agent may be administered wherein the chelating agent sequesters the metal or other ion or binding cofactor, preventing its binding with the attachment moiety and binding moiety.

In another example, if removal of the polymerase from the conductive channel is desired, a molecule that competes with an attachment moiety of a conductive channel for binding to a binding moiety of a polymerase, or competes with a binding moiety of a polymerase for binding to an attachment moiety of a conductive channel, may be added so as to disrupt binding between the attachment moiety and the binding moiety. Thus, where a conductive channel includes an attachment moiety A that binds to a binding moiety B included in a polymerase, an excess of added free molecules of A, not bound to a conductive channel, may outcompete the A moieties of the conductive channel attachment moiety for binding to a B binding moiety of the polymerase. The B binding moieties of the polymerase would then bind to the free A moieties instead of the A moieties of the conductive channel attachment moiety, detaching from the conductive channel. Or, an excess of added free molecules of B, not bound to a polymerase, may outcompete the B moieties of a polymerase binding moiety for binding to an A attachment moiety of the conductive channel. An A attachment moiety of the conductive channel would then bind to a free B moiety instead of a B moiety of a polymerase binding moiety, such that the polymerase detaches from the conductive channel.

After detachment of a polymerase from a conductive channel, whether by removing or chelating a metal ion or other ion or other binding cofactor, or by introducing excess free attachment moiety or binding moiety molecules, a polymerase may subsequently be reattached to a conductive channel. For example, metal ion or other ion or other binding cofactor may be replaced or chelator thereof removed, or free attachment moiety or free binding moiety molecules may be removed, such that upon reintroduction of polymerase including a binding moiety may attach to an attachment moiety of a conductive channel.

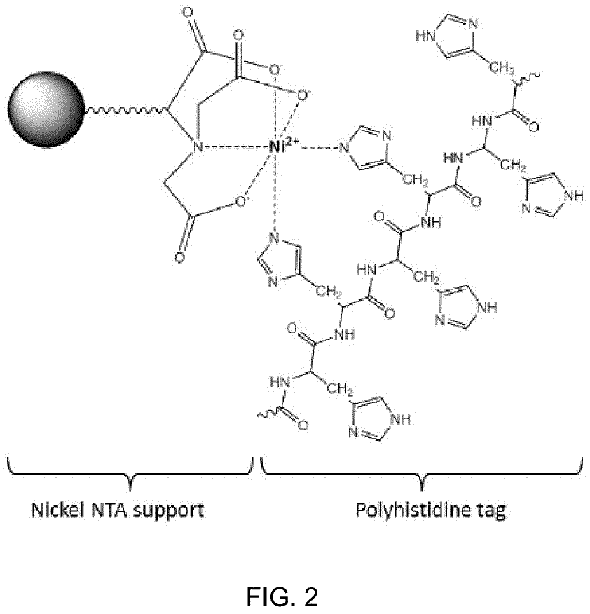

A non-limiting example of an attachment moiety and binding moiety is shown in FIG. 2. In this example, an attachment moiety includes nitrolotriacetic acid (NTA), also called N,N-bis(carboxymethyl)glycine:

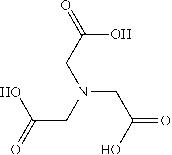 [SMILES: O=C(O)CN(CC(=O)O)CC(=O)O]

NTA, in the presence of nickel ions, forms an attachment to polyhistidine, such as a hexapeptide tag containing six consecutive histidine amino acids (6-His). Other examples may contain more or fewer histidine residues as a binding moiety. FIG. 2 shows a support (such as a conductive channel) including an NTA attachment moiety, complexed with a nickel ion (Ni-NTA) bound to a 6-His binding moiety. In accordance with such an example, a conductive channel may include one or more NTA moieties and polymerase molecules may include a histidine tag such as a 6-His tag. As disclosed herein, one or more attachment moieties such as NTA moieties may be bound to a conductive channel, covalently or non-covalently, for binding to one or more binding moieties such as histidine residues bound, covalently or non-covalently, to a polymerase.

In this example, Ni-NTA binding to a 6-His tag may be disrupted in the presence of a nickel chelator such as EDTA. Chelation of nickel by addition of EDTA breaks the attachment of NTA to 6-His leading to detachment of polymerase from a conductive channel. In another example, excess free imidazole or free compounds including an imidazole group may be added. Histidine includes an imidazole group, which binds to Ni-NTA as shown in FIG. 2. Excess free imidazole may outcompete the histidine imidazole groups for binding to Ni-NTA of the conductive channel, detaching polymerase from the conductive channel.

In some applications, an increased number of attachment moiety-binding moiety bonds per polymerase may be desirable so as to strengthen binding of a polymerase to a conductive channel. An increased number of attachment moieties per conductive channel may allow for stronger attachment of a polymerase to the conductive channel such that the probability of unintentional or unwanted detachment is minimized. One way to achieve this may be to indiscriminately increase the number of attachment moieties individually attached to a conductive channel. In an example where attachment moieties such as NTA moieties are individually bound to a conductive channel, simply increasing the density of NTA moieties bound to a conductive channel could result in more points of attachment for a binding moiety such as a polyhistidine tag to the conductive channel.

However, there may be several disadvantageous drawbacks to such a method. One drawback may be that independently binding single attachment moieties such as NTA to a surface of a conductive channel may result it NTA moieties that are spatially separated from each other such that they do not contribute to increase strength of binding of a histidine tag. NTA moieties that are spatially separated from each other in this manner may each bind to a histidine tag of a polymerase, but not permit the binding of multiple NTA moieties per conductive channel to bind to a given histidine tag of a polymerase. As a consequence, the intended benefit of strengthening the bond between a polymerase and a conductive channel would not be attained or would not be maximized merely by increasing a number of individually bound attachment moieties, such as NTA, per conductive channel.

Another possible disadvantage of attempting to increase strength of binding of a polymerase to a conductive channel by simply increasing a number of attachment moieties individually bound per conductive channel may be a loss of control of a number of polymerase molecules that may bind per conductive channel. In some examples, it may be desirable to have only one polymerase molecule bound per conductive channel. Determining the identity of one or more template molecules by a conductive channel, as explained above, may result from a polymerase associating a nucleotide with a template and attaching the nucleotide to a nascent strand. A charge tag on the nucleotide may be sensed by the conductive channel, modifying the flow of current through the channel. In some examples, different types of nucleotides may have charge tags that differ from each other such that the conductive channel responds differently depending on which type of nucleotide is being incorporated in the growing nascent strand by the polymerase. By extrapolation, detecting the type of nucleotide being incorporated in this manner permits identification of the complementary nucleotide of the template.

For such a method, if more than one active polymerase is bound per conductive channel, there may be increased noise in detection of nucleotide incorporation. If there are two or more active polymerases bound per conductive channel, each may bind a template molecule and catalyze formation of a complementary nascent strand. In such an example, one polymerase may be incorporating one type of nucleotide complementary to one nucleotide of a template while another polymerase is incorporating another type of nucleotide complementary to another nucleotide of another template molecule. The conductive channel may detect both nucleotides, or the charge tags of the nucleotides may interfere with each other and leading to an inaccurate reading or conflicting readings by the conductive channel. For avoidance of such an outcome, it may be desirable to prevent more than one polymerase from binding per conductive channel. If multiple attachment moieties, such as NTA, independently bind to a conductive channel, some may bind to different polymerase molecules from each other, rather than all such attachment moieties bound per conductive channel binding to the same polymerase. More than one polymerase may therefore bind per conductive channel rather than, whereas the intended strengthening of the bond between the polymerase and the conductive channel may not be attained or may be minimized.

Disclosed herein is a conductive channel, and method for making and using such a conductive channel, wherein the conductive channel includes multiple attachment moieties bound thereto in a complex. A complex of attachment moieties attached per conductive channel may overcome disadvantages of multiple attachment moieties individually attached to a conductive channel disclosed above. In an example, a single complex of multiple attachment moieties may be bound per conductive channel. in this manner, multiple attachment moieties in a complex of attachment moieties may be present in sufficient proximity to each other such that they will collectively bind to a binding moiety or binding moieties of the same polymerase as each other. Binding of a complex of attachment moieties to a conductive channel may thereby attain a benefit of strengthening biding of a polymerase to a conductive channel. Furthermore, controlling a number of complexes of attachment moieties bound per conductive channel may minimize, or in one instance entirely prevent, undesirable binding of too many polymerases or more polymerases than is desirable to a conductive channel.

Although one active polymerase per conductive channel may be desirable in some examples, in other examples it may be desirable for more than one polymerase to bind per conductive channel. For example, under conditions when only one template molecule is available for a polymerase reaction per conductive channel, a risk of interference from multiple polymerase reactions per conductive channel to which more than one polymerase molecule is bound may be minimized or avoided. And, in some examples, it may be desirable to have more than one polymerase bound per conductive channel. For example, where only one template molecule is available for a polymerase reaction per conductive channel, it may be desirable to have more than one active polymerase bound per conductive channel to increase the likelihood of the template molecule binding to a polymerase for a polymerase reaction to occur. In other examples, more than one polymerase binding to a conductive channel may be desirable, such as where there is a pool of polymerases and a proportion of polymerases may be inactive or of low efficiency or processivity. In such cases, binding of more than one polymerase per conductive channel may be desirable for attaining a desired level of processivity per conductive channel without sacrificing signal to noise ratio. As will be clear from the following, controlling a number of complexes of attachment moieties per conductive channel more than one polymerase per conductive channel in accordance with the present disclosure may permit binding of one or more than one polymerase per conductive channel as may be desirable for a given circumstance. For example, one, two, three, four, five, or more polymerase molecules may be bound to a conductive channel in accordance with the present disclosure, in a controlled manner, but controlling the number of attachment moiety clusters bound per conductive channel, whereas each cluster may result in increased binding strength per polymerase by nature of its including a number of attachment moieties in sufficiently close proximity to each other that they each, or most of them, bind to a binding moiety or binding moieties of the same polymerase as each other.

A complex of attachment moieties may be bound to a conductive channel by building a branched tree or dendrimer-like structure. In an example, an NTA moiety may be bound to a conductive channel, as a first generation of NTA. A second generation of NTA moieties may be attached to the carboxylic acid groups of the first generation NTA. From the single attachment point of the first generation of NTA, addition of the second generation of NTA results in three potential Ni-NTA attachment sites for a histidine tag. A third generation of NTA moieties may then be attached to the carboxylic acid groups of the second generation NTA. From the single attachment point of the first generation of NTA, addition of the second then third generation of NTA results in nine potential Ni-NTA attachment sites for a histidine tag. Further generations of NTA could then be added to the last generation, thereby progressively increasing the number of attachment moieties per conductive channel, in a cluster to enhance strength of binding of a polymerase. Four, five, or more generations of NTA may be added, yielding 27, 81, or additional tripling of NTA moieties per attachment moiety complex.

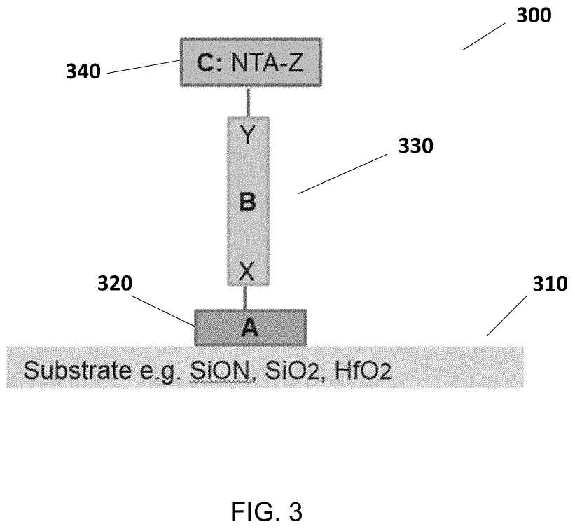

Any suitable method for attaching a first generation NTA to a conductive channel may be used. An illustrative example of an attachment moiety attached to a solid substrate 300, such as a surface of a conductive channel, is depicted in FIG. 3. A substrate 310 may be a surface of a conductive channel or modified surface of a conductive channel. A variety of materials may be included in a surface of a conductive channel to which a polymerase may be attached, including possible components of a surface channel as recited and described above. Examples may include silicon oxynitride (SiON), silicon dioxide (SiO₂), or hafnium dioxide (HfO₂).

A surface of substrate 310 may be modified by a surface modifier 320 which allows attachment of a linker 330 to surface 310. A linker 320 may have a reactive functional group on each end thereof, such as at proximal end X proximal to the substrate 310 and distal end Y distal to the substrate. Reactive group X of linker 330 may be selected so as to be reactive with surface modifier 320. An attachment moiety 340 may then be attached to linker 330. Attachment moiety 340 may have a reactive group Z that may be reactive with distal reactive group Y of linker 330. By modifying surface 310 with surface modifier 320, linker 330 may be attached thereto, and attachment moiety 340 attached to linker 330, creating a bridge from attachment moiety 340 to surface 310. In some examples, there may be no linker 330, and reactive group Z attached to the attachment moiety 340 may instead directly bind to a linker 320 of a conductive channel 310. Many suitable pairings of surface modifier 320 and proximal reactive group X of linker 330, and distal reactive group Y of linker 330 and reactive group Z attached to attachment moiety 340, may be used. A non-exclusive listing of possible pairings is given in Table 1.

TABLE 1
Non-limiting examples of pairings of reactive groups for attaching an
attachment moiety to a conductive channel

| | | | | Attachment |
|---|---|---|---|---|
| | | Linker | | moiety |
| Surface modifier | | Proximal | Distal | reactive |
| End functional | | reactive | reactive | group |
| group | Examples | group X | group Y | Z |
| Amine | APTMS or APTE | NHS | Maleimide | thiol |
| | Azidosilane + phosphine | aldehyde | Thiol | maleimide |
| | (Staudinger azide reduction) | | Alkyne | azide |
| | | | Azide | Alkyne, |
| | | | | DBCO |
| | | | DBCO | azide |
| | | | Tetrazine | TCO |
| | | | TCO | tetrazine |
| Azide | C3- or C11-azidosilane | Alkyne | NHS | amine |
| | Azidolysis of epoxy or | DBCO | aldehyde | Amine, |
| | bromosilane by NaN3 | | | hydrazine, |
| | | | | hydrazide |
| | APTMS or APTES + NHS-azide | | Amine, | aldehyde |
| | | | hydrazine, | |
| | | | hydrazide | |
| | | | maleimide | thiol |
| | | | thiol | maleimide |
| | | | tetrazine | TCO |
| | | | TCO | tetrazine |
| Tetrazine | APTMS or APTES + NHS- | TCO | NHS | amine |
| | tetrazine | | maleimide | thiol |
| | | | aldehyde | Amine, |
| | | | | hydrazine, |
| | | | | hydrazide |
| | | | Amine, | aldehyde |
| | | | hydrazine, | |
| | | | hydrazide | |
| | | | thiol | maleimide |
| | | | azide | Alkyne, |
| | | | | DBCO |
| | | | DBCO | azide |
| Hydrazine or | APTMS or APTES + Cyanuric | aldehyde | NHS | amine |
| hydrazide | chloride + hydrazine | | | |
| | APTMS or APTES + NHS- | | Maleimide | thiol |
| | hydrazide | | | |
| | C3- or C11-azidosilane + | | Thiol | maleimide |
| | alkyne-hydrazide | | | |
| | APTMS or APTES + Succinic | | Alkyne | azide |
| | anhydride or glutaric anhydride + | | Azide | Alkyne, |
| | carbohydrazide + EDC | | | DBCO |
| | | | DBCO | azide |
| | | | Tetrazine | TCO |
| | | | TCO | tetrazine |
| NHS | APTMS or APTES + Succinic | none | none | amine |
| | anhydride or glutaric anhydride + | | | |
| | DCC/NHS or EDC/NHS | | | |
| Epoxy | Epoxysilane silane | | | |
| Aldehyde | Aldehyde | | | |
| Isothiocyanate | APTMS or APTES + PDITC | | | |
| Isocyanate | (3- | | | |
| | Isocyanatopropyl)triethoxysilane | | | |

APTMS=(3-Aminopropyl)trimethoxysilane; APTES=(3-Aminopropyl)triethoxysilane; C3-azidosilane=3-azidopropyltriethoxysilane; C11-azidosilane=11-azidoundecyltrimethoxysilane; PDITC=p-Phenylene diisothiocyanate; DBCO=dibenzocyclooctyne; TCO=trans-cyclooctene.

In some examples, a linker 330 may be bound to a surface modifier 320 in one step, followed by attachment of an attachment moiety 340 to the linker 330 in another step. In another example, a linker 330 may be bound to an attachment moiety 340 in one step, then the linker may be bound to a surface modifier 320 in another step. In still another example, a linker 330 may be bound to a surface modifies 320 and an attachment moiety 340 in the same step.

In an example, an amino group may be added to a surface of a conductive channel as a surface modifier by vapor phase silanization with (3-Aminopropyl)triethoxysilane (APTMS). Subsequently, a bifunctional N-Hydroxysuccinimide-polyethylene glycol-maleimide (NHS-PEGₙ-maleimide) linker may be incubated to allow reaction of the NHS to react with and form a bond with the amino group on the conductive channel surface, thus resulting in attachment of the maleimide group of the NHS-PEGₙ-maleimide linker to the surface of the conductive channel. Subsequent incubation of thiol-NTA would then result in a thiol-maleimide reaction, leading to covalent attachment of an NTA group to the NHS-PEGₙ-maleimide linker as the first generation NTA. In some examples, co-incubation of the amidated conductive channel surface with both NHS-PEGₙ-maleimide linker and thiol-NTA may be performed, to reduce the number of processing steps.

A second generation of NTA can then be added to the first generation of NTA by activating the carboxylic acid groups of NTA with carbonyldiimidazole (CDI), resulting in attachment of three imidazole groups per NTA. Subsequent incubation with NTA-amine results in nucleophilic displacement of the imidazole groups and occupation of each imidazole site by another NTA. Thus, from the first generation NTA, a second generation of NTA may be formed, including a complex of three NTA attachment moieties. Example schemes representing the foregoing steps are shown below.

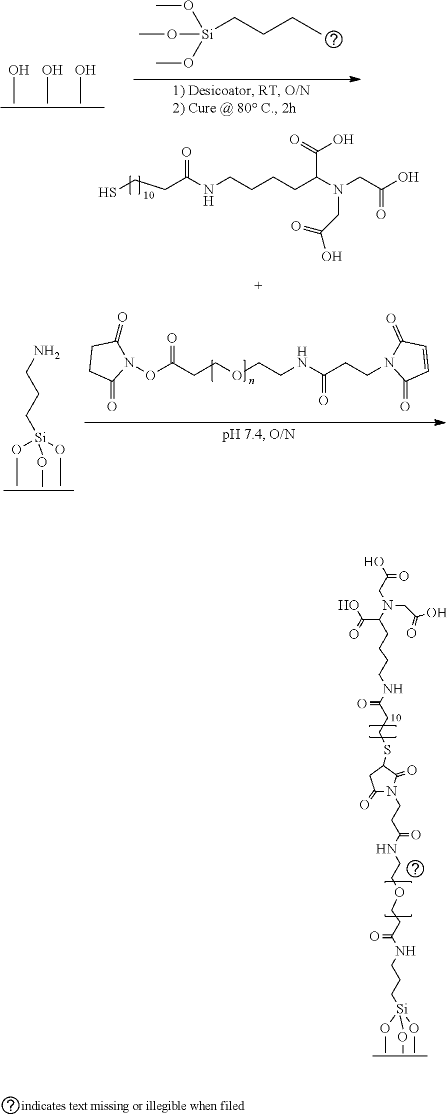

In this example, as described above, vapor phase silanization with (3-Aminopropyl)triethoxysilane (APTMS) adds an amine group to hydroxy groups on the surface of a conductive channel. Single-pot incubation with NHS-PEGₙ-maleimide linker and thiol-NTA results in attachment of first generation NTA to the surface of the conductive channel.

Addition of the second generation of NTA can then be performed as described above and illustrated below.

### 1) CDI Activation of Carboxylic Acid Group

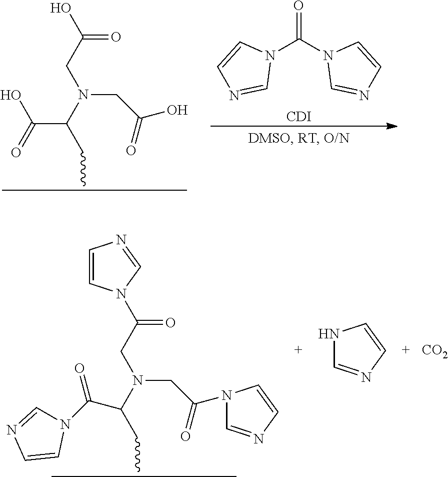

2) Nucleophilic Displacement of Imidazole with NTA-Amine

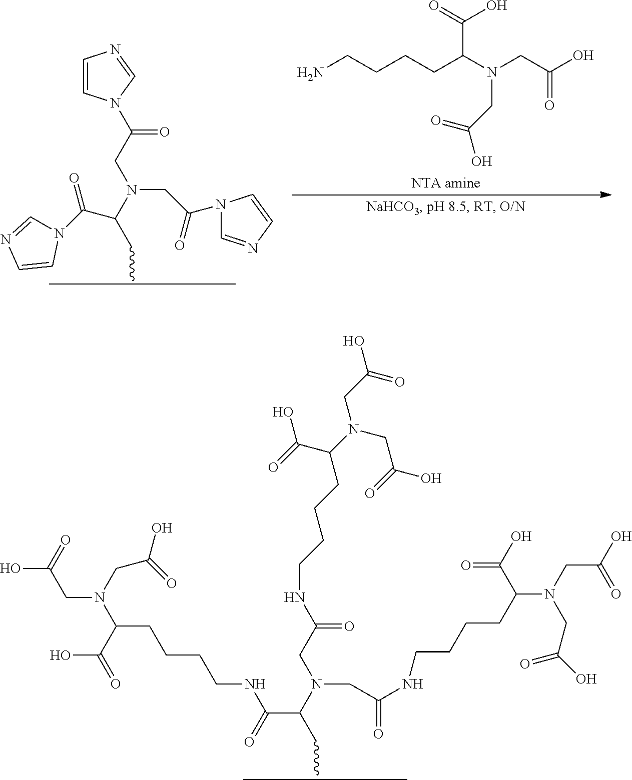

In step 1) above, the carboxylic acid groups of the first generation NTA are activated by overnight incubation with CDI in DMSO at room temperature, releasing an imidazole group and carbon dioxide and attaching an imidazole group per carboxylic acid group of the first generation NTA. In the second step 2), NTA-amine is reacted in the presence of NaCO₃ at pH 8.5 at room temperature overnight, leading to displacement of the imidazole group by the amine of the NTA amine and resulting in attachment of three second generation NTA groups to the first generation NTA group. In this example, repeating steps 1) and 2) above results in formation of the second generation NTA, yielding a complex of nine NTA attachment moieties.

The generation of second, third, and subsequent generations of NTA can also be accomplished with carbodiimides crosslinking chemistry. For example, the carboxylic acid group on a first generation of NTA can be activated using dicyclohexylcarbodiimide (DCC), N,N′-diisopropylcarbodiimide (DIC), or 1-ethyl-3-(3-dimethylaminopropyl)carbodiimide (EDC), in presence of NHS or sulfo-NHS, to form semi-stable amine-reactive NHS ester, which can then be reacted with amine-NTA to generate second generation NTA. Such process could be repeated to form multi-generations of NTA as disclosed herein for formation of an attachment moiety complex. Different combinations of the foregoing chemistries for adding generations of NTA may be used such as where one type of chemistry is used to add one generation of NTA to a prior generation of NTA, and a different chemistry could be used to form a subsequent generation of NTA.

Modifying a number of attachment moiety complexes per conductive channel may be accomplished by modifying a number of first generation NTA groups added. For example, in the above schemes, the number of maleimide groups added may be modified by incubation with NHS-PEG. monofunctional linker spiked with varying amounts of NHS-PEGₙ-maleimide bifunctional linker. With lower concentrations of NHS-PEGₙ-maleimide bifunctional linker, more amine groups would react with and bind to PEG molecules lacking maleimide groups, which would therefore not react bind with a thiol group during a subsequent incubation with thiol-NTA, resulting in lower numbers of NTA attachment moiety complexes becoming attached per solid substrate or conductive channel. Conversely, increasing the concentration of NHS-PEGₙ-maleimide bifunctional linker, relative to NHS-PEG. mono-functional linker, results in more amine groups occupied by PEG molecules bound to maleimide groups and therefore higher numbers of NTA attachment moiety complexes per solid substrate or conductive channel following subsequent incubation with thiol-NTA.

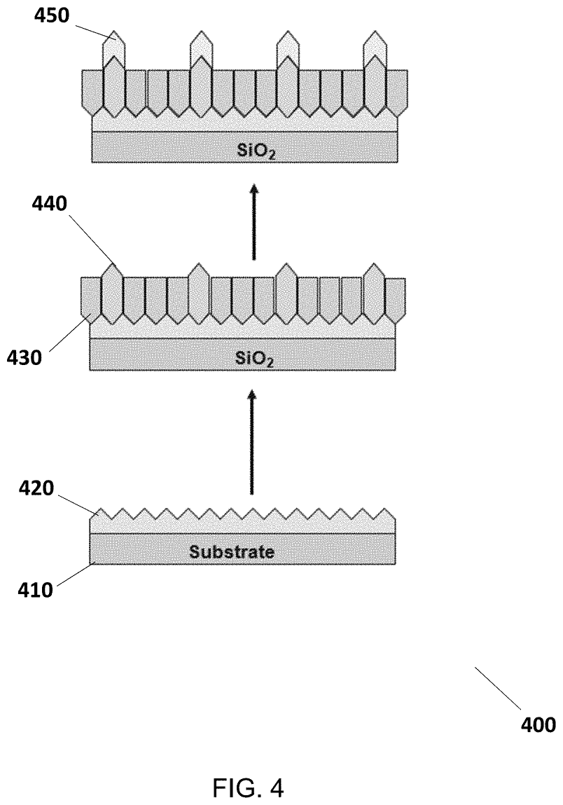

A process for attaching an attachment moiety to a conductive channel 400 is shown in FIG. 4. A surface of a substrate 410 such as a solid surface of a conductive channel is silanized and a reactive group added as a surface modifier 420. Surface modifier 420 may then be reacted with a mixture of monofunctional linker molecules 430 and bifunctional linker molecules 440. A monofunctional linker 430 and a bifunctional linker 440 may each have a proximal functional group that may react with surface modifier 420 to form a bond and attachment to substrate 410. Bifunctional linker 440 may further have a distal functional group that may react with a reactive group of an attachment moiety molecule 450 so as to form a bond and attachment thereto, which distal functional group may be absent from monofunctional linker 430. By varying the relative concentrations of monofunctional linker 430 and a bifunctional linker 440 included in a reaction with surface modifier 420, different concentrations or numbers of initial attachments of an attachment moiety to surface 410 of a conductive channel may be obtained upon which to further develop attachment moiety complexes.

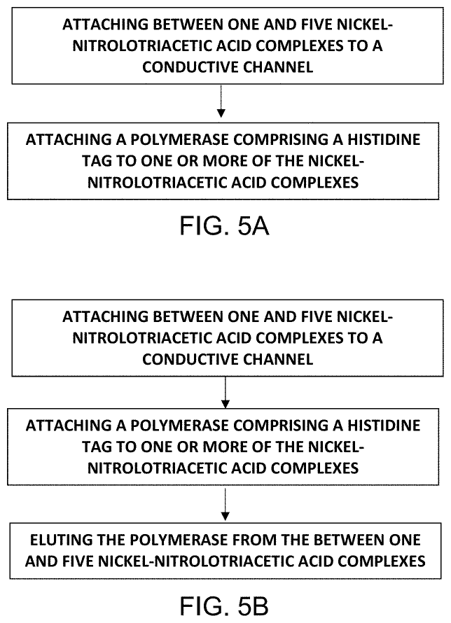

An example of a workflow of a method in accordance with aspects of the present disclosure is presented in FIG. 5A. In this example, between one and five nickel-nitrolotriacetic acid complexes is attached to a conductive channel. The conductive channel is to detect incorporation of a nucleotide including a charge tag into a nascent polynucleotide by the polymerase. The example further includes attaching a polymerase including a histidine tag to one or more of the nickel-nitrolotriacetic acid complexes. Another example is shown in FIG. 5B. In this example, between one and five nickel-nitrolotriacetic acid complexes is attached to a conductive channel. The conductive channel is to detect incorporation of a nucleotide including a charge tag into a nascent polynucleotide by the polymerase. The example further includes attaching a polymerase including a histidine tag to one or more of the nickel-nitrolotriacetic acid complexes. The example further includes eluting the polymerase from the between one and five nickel-nitrolotriacetic acid complexes. Eluting may include chelating nickel with ethylenediaminetetraacetic acid or imidazole.

In some examples, where NHS-PEG. monofunctional linker is included in a reaction step with NHS-PEGₙ-maleimide bifunctional linker, a ratio of NHS-PEGₙ-maleimide bifunctional linker to NHS-PEG. monofunctional linker may be from anywhere between 1:5 to 1:100,000.

As further disclosed herein, linkers of different lengths between an attachment moiety and a conductive channel surface may be advantageously achieved. As a non-limiting representative example, an NHS-PEGₙ-maleimide bifunctional linker may include a larger number of PEG residues to lengthen the distance between an attachment moiety such as NTA and a surface of a conductive channel, whereas fewer PEG residues could be included to shorten the distance between the attachment moiety and a surface of a conductive channel. For example, for NHS-PEGₙ-maleimide, n may be any number from between 0 to about 200, including from 0 to about 24, or about 5, about 6, about 7, about 8, about 9, about 10, about 11, about 12, about 13, about 14, about 15, about 16, about 17, about 18, about 19, about 20, about 21, about 22, about 23, about 24, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, or about 200. The PEG length can be different in the NHS-PEGₙ-maleimide bifunctional linker and the NHS-PEG. monofunctional linker. A distance between a polymerase and conductive channel may be selected based on a number of features, including a desired mobility of an attached polymerase or a desired distance of a polymerase reaction from a conductive channel. As to the latter, the length or distance a charge tag extends from a nucleotide for incorporation into a nascent strand by a polymerase may be related to a preferred distance of a polymerase from a conductive channel, for example. The distance of polymerase from a conductive channel can be between about 1 nm to about 20 nm, including about 3 to about 10 nm, or about 1 nm, about 2 nm, about 3 nm, about 4 nm, about 5 nm, about 6 nm, about 7 nm, about 8 nm, about 9 nm, about 10 nm, about 11 nm, about 12 nm, about 13 nm, about 14 nm, about 15 nm, about 16 nm, about 17 nm, about 18 nm, about 19 nm, or about 20 nm.

In other examples, a linkage between a conductive channel and an attachment moiety may include chemical features that enhance or promote proximal association of a charge tag of a nucleotide with the conductive channel during polymerization, to enhance or improve detection thereof by the conductive channel. In some examples, a charge tag is attached to a nucleotide for incorporation into a nascent strand by a polymerase, based on complementarity to a template strand, by a linkage. Furthermore, the attachment between an attachment moiety and a conductive channel may be, include, or be referred to as a tether. To increase, enhance, or promote proximal association of a charge tag with a conductive channel, such as to enhance detection of the charge tag by the conductive channel, the linkage of the nucleotide and the tether may include chemical features that have an electrostatic attraction to each other.

For example, a tether may include a polynucleotide sequence referred to as an acceptor region, and a linkage between a charge tag and a nucleotide may include a polynucleotide sequence referred to as a specificity region. An accepter region may further be complementary or somewhat complementary to a specificity region, such as by including nucleotides in a sequence that may hybridize. In such an example, during incorporation of a charge-tagged nucleotide to a nascent strand during a sequencing or other process, electrostatic attraction between a specificity region and an acceptor region may serve to bring the charge tag into close proximity with the conductive channel to promote, enhance, strengthen, or otherwise benefit detection of the charge tag by the conductive channel. Examples of pairing chemistries for inclusion in a specificity region and an acceptor region, including complementary nucleotides (e.g., sequences of A, T, G, or C, or inosine, a universal base that can pair with all four native nucleotides of DNA) are disclosed elsewhere such as in International Patent Application PCT/US/2019/018565, the entire contents of which in incorporated herein by reference.

As would be appreciated, other attachment chemistries are available for attaching an attachment moiety to a conductive channel. The amine-NETS and maleimide-thiol examples given above are but representative examples, and other known attachment chemistries could be used, such as those disclosed in Table 1 above, or others. Any of these or other, equally suitable attachment chemistries could be used to attach attachment moieties to a surface of a conductive channel.

The number of surface anchoring points can be controlled by spiking in NETS-PEG that lacks maleimide group to create NTA. This gives the first generation Ni-NTA surface with controllable NTA functional groups (FIG. 3). The 2nd generation NTA surface is created by first carbonyl diimidazole (CDI) activation of the carboxylic acid groups from 1st generation NTA, followed by reaction with a amine-NTA (FIG. 4). This can be repeated to create 3rd or even higher generation of dendrimeric NTA surface.

The examples set forth below and recited in the claims can be understood in view of the above definitions.

### EXAMPLES

The following illustrate particular non-limiting examples in accordance with aspects of the present disclosure, but are by no means intended to limit the scope thereof.

In an example, one, two, or three generations of NTA were formed on a substrate as test attachment moiety complexes as disclosed then bound to green fluorescent protein (GFP) containing 6-His as binding moiety as disclosed herein (with a linker including PEG₂). Then fluorescence was quantified following different durations of storage at room temperature for up to one week. To attach 6-His-tagged GFP, the NTA surface was briefly washed with 40 mM NaOH or 100 mM NaHCO3, then water, ensure COOH deprotonated. To load Ni, surface was incubated in 1% NiSO4 at room temperature for 30 min or 1 hr, followed by water wash 3×, then wash 2× with protein immobilization buffer (HEPES buffer: 50 mM HEPES, pH 7.5, 500 mM NaCl, 0.05% tween-20). The high salt concentration and detergent helps to reduce nonspecific binding of his tag protein. Surface is then incubated with 6-His-GFP at desired concentration (e.g. 1 or 10 ug/mL) in immobilization buffer for 1 hr, followed by buffer wash 5×.

Fluorescence in GFP was imaged under Typhoon scanning using FITC channel. Concentration of GFP was quantified by first eluting GFP off surface using imidazole (100-500 mM) or EDTA (100-500 mM), then measuring the fluorescence intensity of the eluted GFP in a plate using a plate reader, with a calibration curve made with known concentration of GFP.

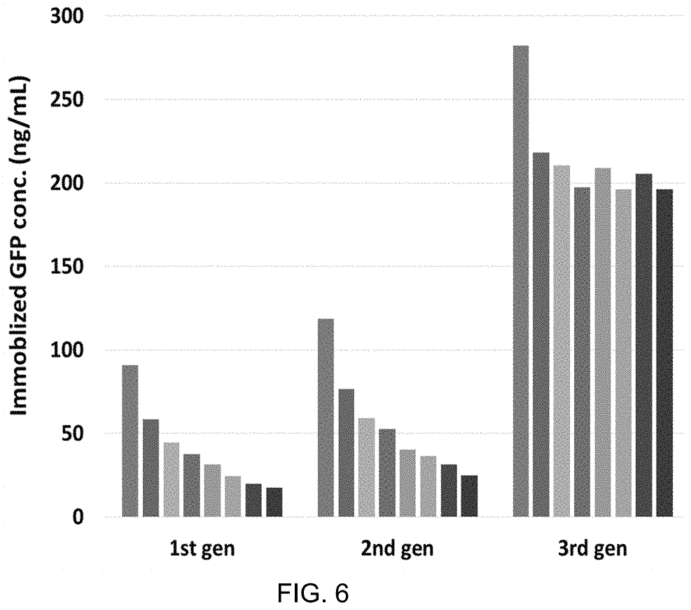

Results are shown in FIG. 6. GFP levels were measured upon attachment to one generation of NTA (left), two generations of NTA (middle), or three generations of NTA (right). Within each set, fluorescence was measured after varying durations of time following washout with immobilization buffer, as follows (presented from left to right for each set of bars in the histograms presented in FIG. 6): 0 hr, 1 hr, 3.5 hr, 5 hr, 1 day, 2 day, 5 day, and 7 day. An attachment moiety complex including a third generation of Ni-NTA caused resulted in the highest binding of 6His-GFP which was also the most stable over a period of 7 days. The immediate drop from between 0 hr and 1 hr was likely due to small percent of unstably bound GFP with levels present from 2 hr to 7 days representing stable, specific binding.

Although examples have been depicted and described in detail herein, it will be apparent to those skilled in the relevant art that various modifications, additions, substitutions, and the like can be made without departing from the spirit of the present disclosure and these are therefore considered to be within the scope of the present disclosure as defined in the claims that follow.

It should be appreciated that all combinations of the foregoing concepts and additional concepts discussed in greater detail herein (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

What is claimed is:

1. A device, comprising:
a conductive channel and a number of polymerase molecules attached to said conductive channel, wherein the number is between one and five and the conductive channel is to detect incorporation of a nucleotide comprising a charge tag into a nascent polynucleotide by the polymerase, and
each of the one or more polymerase molecules comprises a histidine tag, the conductive channel comprises a nickel-nitrolotriacetic acid complex, and the histidine tag is bound to the nickel-nitrolotriacetic acid complex.

2. The device of claim 1, wherein the number of polymerase molecules attached to said conductive channel is less than five.

3. The device of claim 1 wherein the number of polymerase molecules attached to said conductive channel is less than four.

4. The device of claim 1 wherein the number of polymerase molecules attached to said conductive channel is less than three.

5. The device of claim 1 wherein the number of polymerase molecules attached to said conductive channel is one.

6. The device of any one of claims 1 through 5, wherein the nickel-nitrolotriacetic acid complex comprises nine nickel-nitrolotriacetic acid groups.

7. The device of any one of claims 1 through 6, wherein the conductive channel comprises a nanowire having a diameter of between about 10 nm and about 100 nm and a length of between about 50 nm and about 300 nm.

8. The device of claim 7 wherein the nanowire has a diameter of about 30 nm and a length of between about 100 nm and about 150 nm.

9. The device of any one of claims 1 through 8, wherein a surface of the conductive channel further comprises a plurality of polyethylene glycol moieties not directly bound to a complex of nitrolotriacetic acid groups.

10. A method, comprising
attaching between one and five nickel-nitrolotriacetic acid complexes to a conductive channel and attaching a polymerase comprising a histidine tag to one or more of the nickel-nitrolotriacetic acid complexes, wherein
the conductive channel is to detect incorporation of a nucleotide comprising a charge tag into a nascent polynucleotide by the polymerase.

11. The method of claim 10, comprising attaching fewer than five nickel-nitrolotriacetic acid complexes to the conductive channel.

12. The method of claim 10, comprising attaching fewer than four nickel-nitrolotriacetic acid complexes to the conductive channel.

13. The method of claim 10, comprising attaching fewer than three nickel-nitrolotriacetic acid complexes to the conductive channel.

14. The method of claim 10, comprising attaching one nickel-nitrolotriacetic acid complex to the conductive channel.

15. The method of any one of claims 10 through 14, wherein the nickel-nitrolotriacetic acid complex comprises nine nickel-nitrolotriacetic acid groups.

16. The method of any one of claims 10 through 15, wherein the conductive channel comprises a nanowire having a diameter of between about 10 nm and about 100 nm and a length of between about 50 nm and about 300 nm.

17. The method of claim 16, wherein the nanowire has a diameter of about 30 nm and a length of between about 100 nm and about 150 nm.

18. The method of any one of claims 10 through 17, wherein a surface of the conductive channel further comprises a plurality of polyethylene glycol moieties not directly bound to a complex of nitrolotriacetic acid groups.

19. The method of any one of claims 10 through 18, further comprising eluting the polymerase from the between one and five nickel-nitrolotriacetic acid complexes, wherein eluting comprises chelating nickel with ethylenediaminetetraacetic acid or imidazole.

20. The method of any one of claims 10 through 19 further comprising reloading the nitrolotriacetic acid moieties with nickel to re-form nickel-nitrolotriacetic acid complexes and binding a polymerase to the re-formed nickel-nitrolotriacetic acid complexes.

21. A method comprising:
detecting incorporation, using a number of polymerases, of one or more nucleotides into one or more nascent polynucleotide strands complementary to one or more template polynucleotide strands, wherein
the one or more polymerases are each attached to a conductive channel,
one or more of the one or more nucleotides comprise a charge tag and the conductive channel is to detect the charge tag during the incorporation, wherein
each of the one or more polymerases comprises a histidine tag, the conductive channel comprises a nickel-nitrolotriacetic acid complex, and the histidine tag is bound to the nickel-nitrolotriacetic acid complex.

22. The method of claim 21, wherein the number of polymerase molecules attached to said solid support conductive channel is less than five.

23. The method of claim 21 wherein the number of polymerase molecules attached to said solid support conductive channel is less than four.

24. The method of claim 21 wherein the number of polymerase molecules attached to said solid support conductive channel is less than three.

25. The method of claim 21 wherein the number of polymerase molecules attached to said solid support conductive channel is one.

26. The method of any one of claims 21 through 25, wherein the nickel-nitrolotriacetic acid complex comprises nine nickel-nitrolotriacetic acid groups.

27. The method of any one of claims 21 through 26, wherein the conductive channel comprises a nanowire having a diameter of between about 10 nm and about 100 nm and a length of between about 50 nm and about 300 nm.

28. The method of claim 27 wherein the nanowire has a diameter of about 30 nm and a length of between about 100 nm and about 150 nm.

29. The method of any one of claims 21 through 28, wherein a surface of the conductive channel further comprises a plurality of polyethylene glycol moieties not directly bound to a complex of nitrolotriacetic acid groups.
